(19) United States
(12) Patent Application Publication
(10) Pub. No.: US 2005/0222007 A1
(43) Pub. Date: Oct. 6, 2005

(54) Pharmaceutical composition and method for treatment or prevention of vascular disease or states of tissue hypoperfusion with hypoxic and/or ischemic consequences

(76) Inventor: Klas Norrby, Goteborg (SE)

Correspondence Address: MATHEWS, SHEPHERD, MCKAY, & BRUNEAU, P.A., 100 THANET CIRCLE, SUITE 306, PRINCETON, NJ, 08540, US

(21) Appl. No.: 10/505,569

(22) PCT Filed: Feb. 27, 2003
(86) PCT No.: PCT/SE03/00329

§ 371 Date: Apr. 18, 2005

(30) Foreign Application Priority Data
Feb. 27, 2002 (SE) 0200598-1

(51) Int. Cl. A61K 38/40

(52) U.S. Cl. 514/6

(57) ABSTRACT

Disclosed is the use of a substance selected from the group consisting of human apo-lactoferrin and/or peptides derivable from human lactoferrin and/or natural metabolites of human lactoferrin and/or functionally equivalent analogues of human apo-lactoferrin for the production of a pharmaceutical composition for treatment and/or prevention of a vascular disease and/or states of tissue hypoperfusion with hypoxic and/or ischemic consequences. Also a method for treatment of a vascular disease and/or states of tissue hypoperfusion with hypoxic or ischemic consequences wherein a therapeutically effective amount of a substance selected from the group consisting of human apo-lactoferrin and peptides derivable from human lactoferrin and natural metabolites of human lactoferrin and functionally equivalent analogues of human apo-lactoferrin is administered to a patient in need of said treatment is disclosed.

### FIELD OF THE INVENTION

The present invention relates to the use of specific substances for the production of a pharmaceutical composition for treatment or prevention of vascular disease or states of tissue hypoperfusion with hypoxic and/or ischemic consequences. The invention also relates to a new method for treatment or prevention of vascular disease or states of tissue hypoperfusion with hypoxic and/or ischemic consequences.

### BACKGROUND OF THE INVENTION

Lactoferrin (LF) is an 80 kD iron binding glycoprotein which mainly is synthesised by myelopoetic blood cells, such as neutrophil leucocytes, and secretory epithelial cells. LF has been identified in secretions, such as saliva, tears, bronchial secretions, urine, sperm, bile and pancreatic juice. Human milk contains approximately 1 g LF / litre and colostrum (the first breast milk) approximately 7 g LF / litre. LF in breast milk is mainly of apo type, i.e. iron-unsaturated or iron-free LF. LF is also found in plasma but in considerably lower concentration compared to in secretion. LF is furthermore found in specific granulae in neutrophilic leukocytes, from where it is liberated as a response to inflammation.

LF has high affinity to receptors in various specific cell types, including the endothelial cells of the blood vessels. When iron binds to LF the crystallographic structure, and probably also the binding sites, of the molecule is changed, and thus also its function.

LF is considered to have many functions related to the defence of the host, especially with regards to the immunological defence and antibacterial activity.

Bovine apo-LF (apo-bLF) and lactoferricin (bLFcin) have after subcutaneous and per oral administration, with somewhat differing results, been shown to reduce the formation of both primary and daughter tumours in several experimental tumour systems in both mouse and rat. The mechanisms for this anti-tumour effect are not completely clear.

Angiogenesis, or as it is also called neovascularisation, is at the beginning a purely microvascular reaction resulting in the formation of new microvessels from already existing microvessels (the smallest venules and capillaries, respectively). The diameters of the newly formed vessels may increase with time. Angiogenesis is a prerequisite for the growth and metastatic spread of tumours.

Recently the inventor et al. reported for the first time that apo-bLF significantly suppresses VEGF₁₆₅ induced angiogenesis [Norrby K, Mattsby-Baltzer I, Innocenti M, Tuneberg S. (2001) Orally administered bovine lactoferrin inhibits VEGF₁₆₅-mediated angiogenesis in the rat. Int J Cancer 91: 236-240].

Such a suppression of angiogenesis could be the reason behind the above-mentioned inhibitory effects of bLF in tumour models.

VEGF₁₆₅ is a basic and heparin binding, 45 kD homodimeric glycoprotein. It is a unique endothelial specific mitogen and a pro-angiogenic molecule produced by most cell types, in particular during hypoxia (low oxygen pressure in the tissue) or ischemia (insufficient blood supply causing hypoxia, further discussed below). High affinity receptors for VEGF₁₆₅ are almost exclusively located on endothelial cells, cells that cover the walls of the blood vessels and have the ability to form new blood vessels.

The human gene for VEGF is found on chromosome 6 (6p21.3). Alternative exon splicing of one single VEGF gene results in the formation of four different molecules with 121, 165, 189 and 206 amino acid residues, respectively. VEGF₆₅ is by far the most common isoform. Hypoxia, and glucose deficiency connected therewith, is the most efficacious factor for upregulation of VEGF. Hypoxia-inducible factor, HIF, is a key regulator of responses to hypoxia inducing the VEGF gene. Also the number of specific VEGF receptors on the endothelial cells is increased during hypoxia.

During insufficient local blood supply, ischemia, both hypoxia and inflammation occurs. Impaired blood circulation, ischemia, can cause serious clinical symptoms in e.g. the cardiac muscle resulting in angina pectoris and/or myocardial infarction, in the brain resulting in stroke or cerebral infarction, or in the lower limbs resulting ultimately in gangrene.

Significant physiologic angiogenesis occurs hardly ever in adults, with the exception for in the ovaries, the endometrium, and the placenta. Angiogenesis is however a rapid, life sustaining reaction in wound healing and inflammation. There is thus an exceptionally powerful regulation of the balance between pro- and anti-angiogenic factors in the body. The balance is upheld by an apparently large, not yet fully elucidated, system of pro- and anti-angiogenic endogenous molecules.

### SUMMARY OF THE INVENTION

As stated above, it was recently reported that bovine apo-LF significantly inhibits VEGF₁₆₅ induced angiogenesis. Very unexpectedly and surprisingly, the inventor has now found, in exactly the same experimental system, that human apo-LF on the contrary stimulates VEGF₁₆₅ induced angiogenesis. Human apo-LF has thus a pro-angiogenic effect, which can be used for therapeutic angiogenesis.

Thus, the present invention relates to the systemic or local use of human apo-lactoferrin and/or peptides derivable from human apo-lactoferrin, natural metabolites thereof and/or functionally equivalent analogues thereof in order to enhance VEGF induced angiogenesis to prevent tissue damage. In vascular disease such tissue damage is typically caused by occlusive or non-occlusive vascular disease leading to tissue hypoxia or ischemia.

The clinical effects of such tissue hypoxia or ischemia may be exemplified by impending or manifested infarction such as myocardial infarction, stroke or gangrene. Another important vascular disease related to such hypoxic/ischemic states is angina pectoris. Other clinical conditions that would benefit from enhanced VEGF induced angiogenesis are various types of wound healing situations such as in peptic ulcers and leg ulcers. Certain types of male hair loss, such as androgenic alopecia, might also be a condition where enhanced angiogenesis might be beneficial.

A common denominator for all these ischemic and/or hypoxic conditions is the resulting local expression or biological effect of VEGF. According to the present invention the use of human apo-lactoferrin and/or peptides derivable from human apo-lactoferrin, natural metabolites thereof and/or functionally equivalent analogues thereof, enhances angiogenesis. An attractive, but not binding, explanation for this is that these substances enhance and/or stimulate the expression or the biological effects of VEGF.

One objective of the present invention is thus the use of a substance selected from the group consisting of human apo-lactoferrin and/or peptides derivable from human lactoferrin and/or natural metabolites of human lactoferrin and/or functionally equivalent analogues of human apo-lactoferrin for the production of a pharmaceutical composition for treatment and/or prevention of a vascular disease or states of tissue hypoperfusion with hypoxic and/or ischemic consequences.

Another object of the present invention is a method for treatment or prevention of a vascular disease or states of tissue hypoperfusion with hypoxic and/or ischemic consequences where in a therapeutically effective amount of a substance selected from the group consisting of human apo-lactoferrin and/or peptides derivable from human lactoferrin and/or natural metabolites of human lactoferrin and/or functionally equivalent analogues of human apo-lactoferrin is administered to a patient in need of said treatment.

The characterizing features of the invention will be evident from the following description and the appended claims.

### DETAILED DESCRIPTION OF THE INVENTION

As stated above, the invention is based on the use of a substance selected from the group consisting of human apo-lactoferrin and/or peptides derivable from human apo-lactoferrin and/or natural metabolites of human apo-lactoferrin and/or functionally equivalent analogues of human apo-lactoferrin.

The lactoferrin used according to the invention may be of any isoform. It may also be recombinant human apo-lactoferrin.

Said natural metabolites may for example be lactoferricin, i.e. a pepsin-cleaved fragment from human lactoferrin or hydrolysates of human lactoferrin.

Said functionally equivalent analogues are substances that are structurally similar to human apo-lactoferrin or to natural metabolites thereof, which have essentially the same pro-angiogenic effect as human apo-lactoferrin.

Said peptides derivable from human lactoferrin may be fragments or modified fragments obtainable from human lactoferrin. They may be either naturally occurring or synthetically produced peptides. They may for example be the peptides described in the International application with publication number WO 00/01730, which is incorporated herein by reference. In particular, the peptides disclosed in the sequence listing of WO 00/01730 are suitable for use according to the present invention. These include peptides constituted of all or some of the amino acids 12-40 of human lactoferrin counted from the N-terminal end, and preferably modified versions thereof. More specifically they include the peptides formed of the sequences constituted of amino acids 16-40 and amino acids 18-40 from the N-terminal end of human lactoferrin, with some alterations, and also sequences with only 14 residues, roughly corresponding to residues 18-31 of human lactoferrin wherein C-20 is replaced by A, Q-22 is replaced by K, and N-26 is replaced by D. They also include peptides formed of the amino acids in positions 12-31, counted from the N-terminal end, in the sequence constituting human lactoferrin, as well as modifications thereof, and also fragments of this sequence consisting of at least 7 amino acids. Furthermore, they include peptides consisting of 11-17 amino acids corresponding to the sequences that begin with one of the amino acids in positions 15-21 and end with the amino acid in position 31, counted from the N-terminal end, in the sequence constituting human lactoferrin, as well as modifications thereof. Moreover, they include peptides consisting of 12 amino acids based on the sequence consisting of the amino acids in positions 20-31 in human lactoferrin, counted from the N-terminal end.

The substances and pharmaceutical compositions can according to the invention be used for all medical disorders that benefit from stimulation of VEGF induced angiogenesis, or diseases where there is an insufficient effect of VEGF either caused by increased effects of anti-angiogenic factors, such as certain hormones, or caused by insufficient production of VEGF. By insufficient effect of VEGF or insufficient production of VEGF is intended that angiogenesis does not occur sufficiently for prevention or improvement of a disease.

For the purpose of this disclosure, the terms “illness”, “disease”, “medical condition”, “abnormal condition” and the like can be used interchangeably with “medical disorder”. Examples of disorders that benefit from the treatment according to the invention, i.e. the enhancement of VEGF stimulated angiogenesis, are conditions of hypoxia and/or ischemia resulting in angina pectoris, impending or manifested myocardial infarction, stroke or gangrene, wounds, such as peptic or leg ulcers that are slow in healing, and certain types of hair loss.

The term “treatment”, as it is used herein, relates to both treatment in order to cure or alleviate a disease or a condition, and to treatment in order to prevent the development of a disease or a condition. The treatment may either be performed in an acute or in a chronic way.

VEGF is upregulated virtually exclusively under conditions of hypoxia and is the key angiogenic factor linking ischemia and collateral compensatory angiogenesis. In hypoxia the degradation of the transcription factor hypoxia-inducible factor (HIF-1 alpha) is suppressed leading to activation of the hypoxia responsive elements and subsequently the VEGF gene. As stated above, the substances and pharmaceutical compositions according to the invention are suitable for treatment of tissue ischemia. VEGF₁₆₅ is over expressed during tissue ischemia, i.e. locally decreased oxygen pressure due to insufficient oxygen supply due in turn to impaired blood circulation. During ischemia the effect of VEGF₁₆₅ is considered to be necessary for local compensatory collateral angiogenesis, which leads to locally increased blood supply. If ischemic conditions are not reversed they can result in infarction, i.e. tissue death. This can happen in e.g. the cardiac muscle (myocardial infarction), the brain (brain infarction or stroke) or in distant parts of the lower limbs, such as toes, the feet or the lower part the leg (gangrene) resulting in severe clinical consequences. Stimulation of the VEGF₁₆₅ mediated angiogenesis with the substances according to the invention reduces the risk of i.a. myocardial infarction, stroke and gangrene.

The substances and pharmaceutical compositions according to the invention are thus suitable for treatment of cardiovascular disease, especially coronary and carotid artery disease. Cardiovascular disease caused by vascular damage, endothelial dysfunction and atherosclerotic changes is the most common cause of death in the western world. Atherosclerosis is in particular common in connection with diabetes mellitus. The occurrence of diabetes, and in particular diabetes type II, increases dramatically globally, probably due to changed ways of life.

Furthermore, the substances and pharmaceutical compositions according to the invention are also suitable for treatment of peripheral artery occlusive disease, PAOD, which is caused by atherosclerosis and affects primarily the lower limbs. Advanced cases of PAOD cannot be treated by reconstructive-, angioplastic- or by pass-surgery. The use of the substances according to the invention may prevent the need of amputation or angiogenic gene therapy in impending gangrene. Current clinical trials with therapeutic angiogenesis is essentially based on the administration of the gene of an angiogenic factor, such as VEGF₁₆₅, with the aid of viral or plasmid vector systems. These techniques have limitations and complications, e.g. it is difficult to achieve the correct dosage and not only endothelial cells within the hypoxic tissue are affected [Ferber, D. (2001) Gene therapy: Safer and virus-free? Science 295:1638-42]. Several gene therapy clinical trials are currently going on worldwide, but so far no such treatment has been approved for clinical use.

The use of the substances or pharmaceutical compositions according to the invention thus provides a new way to treat PAOD. According to the invention it is possible to replace the use of currently tested types of therapeutic angiogenesis with the use of the substances or pharmaceutical compositions according to the invention. One advantage is that this treatment may be given continuously, or repeatedly, during long periods of time without or with limited side effects, since this treatment is targeted in that only ongoing VEGF mediated angiogenesis is stimulated.

The pharmaceutical composition, or medicinal product, according to the invention may also comprise other substances, such as an inert vehicle, or pharmaceutically acceptable adjuvant, carriers, preservatives etc, which are well known to persons skilled in the art.

In the method according to the present invention a therapeutically effective amount of the above substance is administered to the patient. The expression “therapeutically effective amount” relates to an amount that will lead to the desired therapeutic effect, i.e. an amount that will enhance the VEGF mediated angiogenesis.

The substance or pharmaceutical composition according to the invention is administered to the patient orally, parenterally, locally and/or by inhalation. For local administration the pharmaceutical composition or the substance may e.g. be in the form of an ointment, a solution or a spray. The term “patient”, as it is used herein, relates to any human or non-human mammal in need of treatment according to the invention, i.e. any human or non-human mammal that benefits from VEGF induced angiogenesis or therapeutic angiogenesis.

The invention will now be further explained in the following examples. These examples are only intended to illustrate the invention and should in no way be considered to limit the scope of the invention.

EXAMPLES

Methods

The method used herein to study the formation of new microvessels, “the rat mesenteric-window angiogenesis assay”, has the following advantages compared to other models of angiogenesis: (1) Similar to almost all tissues in human adults the test tissue used is natively vascularised and lacks physiological angiogenesis. (2) No surgical procedures, which inevitably cause a wound healing reaction accompanied by angiogenesis, are used. (3) The method allows for a good quantification of the angiogenic response in objective parameters.

In order to induce angiogenesis the animals are given repeated picomolar doses of recombinant VEGF₁₆₅ intra peritoneally, which quickly reaches the test tissue.

When the animal has been sacrificed, the intact test tissue is studied as spread preparations on objective slides, which is a major advantage compared to the study of the presence of vessels in microtome-sectioned tissue. Several of the parameters are measured by interactive image analysis techniques. A number of relevant, objective parameters are measured using light microscopic morphometry with high accuracy. By using purified anti-rat endothelium monoclonal antibody CL 043AP also the smallest microvessels are visualized immunocytochemically [Norrby K, Mattsby-Baltzer I, Innocenti M, Tuneberg S. (2001) Orally administered bovine lactoferrin inhibits VEGF₁₆₅-mediated angiogenesis in the rat. Int J Cancer 91: 236-240]. By using this method, it is possible to administer modulators of angiogenesis systemically, which simulates a clinical therapeutic situation.

A measurement of spatial distribution of the vessel network is “vascularised area” (VA) expressed in % of the whole area per mesenterial window [Norrby K. (1994) Basic fibroblast growth factor and mammalian de novo angiogenesis. Microvasc Res 48: 96-113; Norrby K. (1996) Vascular endothelial growth factor and mammalian de novo angiogenesis. Microvasc Res 51: 153-163].

Microvascular density is measured as “microvascular length”, MVL (percentage pixles occupied by vessels made 1 pixle thick); “total microvascular length” (TMVL)=VA×MVL [Norrby K. (1994) Basic fibroblast growth factor and mammalian de novo angiogenesis. Microvasc Res 48: 96-113; Norrby K. (1996) Vascular endothelial growth factor and mammalian de novo angiogenesis. Microvasc Res 51: 153-163]. Microvascular density and pattern formation is measured in terms of the length of the individual microvascular segments (Le. MS, length of microvessel segment, the distance between two subsequent branching points) and the number of them (No. MS, number of microvessel segments) per volume unit of tissue, as well as the microvessel tortuosity (MVT) [Norrby K. (1998) Microvascular density in terms of number and length of microvessel segments per unit tissue volume in mammalian angiogenesis. Microvasc Res 55:43-53]. The method for measurement of the frequency of microvascular loop formation (In. LF, index of loop formation), intersection (In. IS, index of intersection) and the number of branching points (No. BP) per volume unit of tissue was also used [Näslund I, Norrby K. (2000) NO and de novo mammalian angiogenesis: further evidence that NO inhibits bFGF-induced angiogenesis while not influencing VEGF₁₆₅-induced angiogenesis. APMIS 108: 29-37; Norrby K. (2000) 2.5 kD and 5.0 kD heparin fragments specifically inhibit microvessel sprouting and network formation in VEGF₁₆₅-mediated mammalian angiogenesis. Int J Exp Path 81: 191-198]. Le. MS is influenced by several factors, such as the ability of the microvascular segments to increase their length, to divide and to form interconnecting loops.

The number of microvessel sprouts per unit tissue volume (No. SP), their length (Le. SP) and degree of tortuosity (SPT) were measured at the edge of the network in one of the experiments (Table 4), as described elsewhere [Näslund I, Norrby K. (2000) NO and de novo mammalian angiogenesis: further evidence that NO inhibits bFGF-induced angiogenesis while not influencing VEGF₁₆₅-induced angiogenesis. APMIS 108: 29-37; Norrby K. (2000) 2.5 kD and 5.0 kD heparin fragments specifically inhibit microvessel sprouting and network formation in VEGF₁₆₅-mediated mammalian angiogenesis. Int J Exp Path 81: 191-198]. Increase in No. SP indicates increased microvessel proliferation. The Le. SPs were ranked in order of size in each treatment group.

### Example 1

In this experiment the effect of ingested apo-hLF on VEGF₁₆₅ mediated angiogenesis was demonstrated. The results are shown below in Table 1.

Lactoferrin, dissolved in saline, was given by tube feeding twice daily from Sunday afternoon (Day -1) to Friday afternoon (Day4). Vehicle controls received saline by tube feeding. The angiogenic treatment with VEGF was given i.p. on Days 0-4 (twice daily).

TABLE 1
Effect of orally administered iron-unsaturated human lactoferrin
(20 mg/kg twice daily), apo-hLF, on VEGF₁₆₅ mediated angiogenesis

| | | | | |
|---|---|---|---|---|
| | VEGF treated + | VEGF treated + | % of | Untreated |
| | vehicle | apo-hLF | vehi- | controls |
| Variable | n = 14 | n = 14 | cle | n = 8 |

Microvessel proliferation

| | | | | |
|---|---|---|---|---|
| VA | 7.82 ± 1.93 | 12.09 ± 1.49^{a} | 155 | 1.18 ± 0.50 |
| MVL | 1.085 ± 0.137 | 1.465 ± 0.077^{a} | 135 | 0.28 ± 0.04 |
| TMVL | 8.49 ± 2.10 | 17.72 ± 2.19^{b} | 209 | 0.33 ± 0.14 |
| No. BP | 238.9 ± 39.7 | 376.7 ± 31.7^{b} | 158 | 32.5 ± 6.7 |
| No. MS | 279.0 ± 49.4 | 457.6 ± 40.8^{b} | 164 | 32.7 ± 7.4 |
| Le. MS | 7-17 | 5-15^{d} | \ | 8-19 |
| (0-10) | (range, μm) | | | |
| Le. MS | 178-534 | 157-668^{d} | \ | 265-719 |
| (90-100) | (range, μm) | | | |
| Le. MS | 61.5 | 55.1 | 90 | 78.1 |
| median, | | | | |
| μm | | | | |

Microvessel network pattern formation

| | | | | |
|---|---|---|---|---|
| MVT | 7.39 ± 0.34 (8) | 7.54 ± 0.14 (12) | 102 | 4.70 ± 0.73 |
| In. LF | 1.144 ± 0.014 | 1.208 ± 0.008^{c} | 106 | 0.988 ± 0.031 |
| In IS | 13.89 ± 1.23 | 15.68 ± 0.87 | 112 | 10.20 ± 2.58 |

^{a}p ≦ 0.05;
^{b}p ≦ 0.01;
^{c}p ≦ 0.002;
^{d}p ≦ 0.0001 compared with vehicle controls.
\ indicates statistically significant shortening of the Le. MS.
Figures in parentheses indicate number of animals, if less than 14 (or if less than 8 in the case of untreated controls), analysed according to the set criteria.
The distribution of Le. MS, the (0-10) percentile, the (90-100) percentile, and the median value, was based on thousands of individually measured microvessel segments in both vehicle controls and in the group of animals treated with apo-hLF.
Mean ± SEM.

VA, MVL, TMVL, No. BP, No. MS and MVT increased statistically significantly in the animals receiving VEGF i.p. and saline orally as compared to the untreated controls, thus demonstrating the proangiogenic effect of the VEGF treatment.

Treatment with apo-LF further increased these measurements, indicating an additional stimulatory effect of apo-hLF on VEGF-stimulated angiogenesis.

The results demonstrate that oral administration of apo-hLF significantly enhanced the VEGF mediated angiogenic response.

### Comparative Example 1

In this comparative experiment it was demonstrated that ingested holo-hLF, in contrast to apo-hLF, does not have any significant effect on VEGF₁₆₅ mediated angiogenesis. The results are shown below in Table 2.

Groups and treatments were as described under Example 1, Table 1, apart from that apo-hLF was replaced by holo-hLF.

TABLE 2
Effect of orally administered iron-saturated human lactoferrin
(20 mg/kg twice daily), holo-hLF, on VEGF₁₆₅ mediated angiogenesis.

| | | | | |
|---|---|---|---|---|
| | VEGF-treated + | VEGF-treated + | % of | Untreated |
| | vehicle | holo-hLF | vehi- | controls |
| Variable | n = 14 | n = 14 | cle | n = 8 |

Microvessel proliferation

| | | | | |
|---|---|---|---|---|
| VA | 5.95 ± 0.73 | 5.77 ± 1.12 | 97 | 1.38 ± 0.38 |
| MVL | 1.110 ± 0.079 | 1.067 ± 0.121 | 96 | 0.381 ± 0.053 |
| TMVL | 6.80 ± 0.98 | 6.45 ± 1.20 | 95 | 0.53 ± 0.14 |
| No. BP | 242.6 ± 20.4 | 241.1 ± 31.7 | 99 | 62.4 ± 12.4 |
| No. MS | 286.0 ± 25.1 | 289.0 ± 39.7 | 101 | 68.7 ± 15.6 |
| Le. MS | 5-17 | 5-15^{a} | \ | 5-16 |
| (0-10) | (range, μm) | | | |
| Le. MS | 174-710 | 172-576 | | 221-458 |
| (90-100) | (range, μm) | | | |
| Le. MS | 60.2 | 58.9 | 98 | |
| median, | | | | |
| μm | | | | |

Microvessel network pattern formation

| | | | | |
|---|---|---|---|---|
| MVT | 8.36 ± 0.29 | 9.13 ± 0.34 (12) | 109 | 6.95 ± 0.39 |
| | (13) | | | (3) |
| In LF | 1.174 ± 0.007 | 1.183 ± 0.015 | 101 | 1.054 ± 0.051 |
| In IS | 11.71 ± 1.04 | 13.00 ± 0.96 | 111 | 14.24 ± 1.70 |

p ≦ 0.05 compared with vehicle controls of the same experiment
\ indicates statistically significant shortening of the Le. MS compared with vehicle control
Figures in parentheses indicate number of animals, if less than 14 (or if less than 8 in the case of untreated controls), analysed according to the set criteria.
The distribution of Le. MS, the (0-10) percentile, the (90-100) percentile, and the median value, was based on several thousands individually measured microvessel segments in both vehicle controls and in the group of animals treated with holo-hLF.
Mean ± SEM.

VA, MVL, TMVL, No. BP, No. MS and MVT increased statistically significantly in the animals receiving VEGF i.p. and saline orally as compared to the untreated controls, thus demonstrating the proangiogenic effect of the VEGF treatment.

Unlike with apo-hLF, treatment with holo-hLF did not increase the VEGF-stimulated angiogenesis.

### Comparative Example 2

Besides VEGF, bFGF (basic fibroblast growth factor) is probably the most important angiogenic factor. It was thus of interest to test whether apo-hLF also affected bFGF stimulated angiogenesis.

In this comparative experiment it was demonstrated that apo-hLF does not have any significant effect on bFGF-mediated angiogenesis, in contrast to its effect on VEGF₁₆₅ mediated angiogenesis. The results are shown below in Table 3.

The same experimental conditions were used as in the experiments with VEGF induced angiogenesis.

TABLE 3
Effect of orally administered iron-unsaturated human lactoferrin
(20 mg/kg twice daily), apo-hLF, on bFGF mediated angiogenesis

| | | | | |
|---|---|---|---|---|
| | bFGF-treated + | BFGF-treated + | | Untreated |
| | saline | holo-hLF | % of | controls |
| Variable | n = 14 | n = 14 | vehicle | n = 8 |

Microvessel proliferation

| | | | | |
|---|---|---|---|---|
| VA | 7.79 ± 0.98 | 7.21 ± 0.88 | 93 | 4.49 ± 1.01 |
| MVL | 1.049 ± 0.090 | 1.226 ± 0.068 | 117 | 0.514 ± 0.053 |
| TMVL | 8.17 ± 1.03 | 8.84 ± 1.08 | 108 | 2.30 ± 0.52 |
| No. BP | 252.9 ± 32.4 | 274.8 ± 18.7 | 109 | 86.7 ± 12.7 |
| No. MS | 302.5 ± 42.1 | 322.6 ± 23.4 | 107 | 95.0 ± 15.7 |
| Le. MS | 5-12 | 5-13 | — | 5-18 |
| (0-10) | (range, μm) | | | |
| Le. MS | 159-530 | 172-716^{a} | \ | 192-485 |
| (90-100) | (range, μm) | | | |
| Le. MS | 55.9 | 60.8 | 109 | 70.6 |
| median, μm | | | | |

Microvessel network pattern formation

| | | | | |
|---|---|---|---|---|
| MVT | 8.90 ± 0.17 | 9.35 ± 0.20 | 105 | 8.66 ± 0.43 |
| | (13) | | | (5) |
| In. LF | 1.178 ± 0.014 | 1.168 ± 0.010 | 99 | 1.075 ± 0.027 |
| In IS | 15.13 ± 0.91 | 13.52 ± 1.18 | 89 | 9.16 ± 1.55 |

^{a}p ≦ 0.0001 compared with vehicle control.
/ indicates statistically significant lengthening of the Le. MS.
—indicates statistically insignificant change of Le. MS.
Figures in parentheses indicate number of animals, if less than 14 (or if less than 8 in the case of untreated controls), analysed according to the set criteria.
The distribution of Le. MS, the (0-10) percentile, the (90-100) percentile, and the median value, was based on thousands of individually measured microvessel segments in both vehicle controls and in the group of animals treated with apo-hLF.
Mean ± SEM.

VA, MVL, TMVL, No. BP and No. MS increased significantly in statistical terms in the animals receiving bFGF i.p. and saline orally as compared to the untreated controls, thus demonstrating the pro-angiogenic effect of the bFGF treatment.

It was found (see Table 3) that bFGF mediated angiogenesis is not affected by apo-hLF, apart from the fact that the longer microvascular segments (Le. MS, see above) were significantly lengthened. The meaning of this is not clear, and one should note that the corresponding population or Le. MS were significantly shortened by apo-hLF in VEGF mediated angiogenesis (see Table 1).

These results indicate that enhancing effect of apo-hLF seen in VEGF mediated angiogenesis is specific and not valid for angiogenesis in general, which is very interesting both theoretically and clinically.

### Comparative Example 3

In this comparative experiment it was demonstrated that continuously s.c. infused apo-hLF signficantly enhanced VEGF165-mediated angiogenesis (Table 4) and the number of capillary sprouts (Table 5).

TABLE 4
Effect of subcutaneously continuously infused iron-unsaturated
human lactoferrin, apo-hFL, on VEGF164-mediated angiogenesis.
Vascularized area (VA), microvascular length (MVL) and
total microvascular length (TMVL)

| | | Apo-hLF | |
|---|---|---|---|
| | Vehicle | 2 mg/kg/24 hr | 20 mg/kg/24 hrs |
| Variable | n = 14 | n = 11 | n = 12 |
| VA | 8.04 ± 1.53 | 12.35 ± 2.46 | 9.71 ± 1.60 |
| | (100%) | (154%) | (121%) |
| MVL | 1.004 ± 0.100 | 1.260 ± 0.120a | 1.269 ± 0.113a |
| | (100%) | (125%) | (126%) |
| TMVL | 8.04 ± 1.54 | 15.55 ± 3.09b | 12.32 ± 2.03 |
| | (100%) | (193%) | 153%) |

Lactoferrin, dissolved in saline, was infused s.c. for 7 days, starting on Day - 1, using an Alzet ® osmotic pump. Vehicle controls received saline s.c. by the same type of pump. The angiogenic treatment with VEGF was given i.p. on Days 0-4. The animals were sacrificed on Day 8.
ap ≦ 0.05;
bp ≦ 0.05 compared with vehicle controls. In addition, the difference between control and the combined data of the low and the high dose of apo-hLF was significant in terms of MVL and TMVL (p ≦ 0.05).
Number of animals, n.
Mean ± SEM.

TABLE 5
Effect of subcutaneously continuously infused human iron-unsaturated
lactoferrin, apo-hLF, on VEGF₁₆₄-mediated angiogenesis.
Number of microvessel sprouts per unit tissue volume (No. SP),
sprout length (Le. SP) and degree of sprout toruosity (SPT)

| | | Apo-hLF |
|---|---|---|
| | Vehicle | 2 mg/kg/24 hr |
| Variable | n = 14 | n = 11 |
| No. SP | 107.1 ± 9.3 | 133.4 ± 9.2^{a} |
| | (100%) | (125%) |
| Le. SP, 0-10 percentile | 8-72 | 14-66^{b} (−) |
| (range, μm) | | |
| Le. SP, 90-100 percentile | 482-1237 | 441-1042^{c} (−) |
| (range, μm) | | |
| Le. SP, median, μm | 200.1 | 168.9 |
| | (100%) | (84%) |
| SPT (A.U.) | 10.0 ± 0.3 | 11.0 ± 0.3^{a} |
| | (100%) | (110%) |

Lactoferrrin, dissolved in saline, was infused for 7 days, starting on Day -1, using an Alzet® osmotic pump. Vehicle controls received saline s.c. by the same type of osmotic pump. The angiogenic treatment with VEGF was given i.p. on Days 0-4. The animals were sacrificed on Day 8. The variables were measured at the edge of the microvascular network in those mesenteric windows that were also analyzed in regard to a number of other angiogenesis variables within the same network, as presented in Table 3.

The distribution of Le. SP (the 0-10 percentile, the 90-100 percentile and the median) was based on approximately 1,500 individually measured sprouts in vehicle controls. Statistically significant shortening of the Le. MS compared with vehicle control, (−).

a) p ≦0.05, b) p ≦0.01, and c) p ≦0.0001 compared with vehicle controls.

### Conclusions from the Examples

To summarize, the results presented in the examples and in Tables 1-5 show that the oral or s.c. administration of apo-hLF specifically enhances VEGF₁₆₅ mediated angiogenesis in the rat mesenteric-window angiogenesis assay.

This effect seems to be specific for VEGF mediated angiogenesis since bFGF mediated angiogenesis was not affected by apo-hLF. Also, the stimulatory effect of LF on VEGF mediated angiogenesis seems to be specific for the apo-form since oral administration of holo-hLF did not affect the measured angiogenic parameters.

1-32. (canceled)

33. A method for treatment or prevention of a vascular disease or states of tissue hypoperfusion with hypoxic or ischemic consequences in a patient, the method comprising the following steps:
selecting a substance from the group consisting of human apo-lactoferrin, human lactoferricin, peptides derivable from human lactoferrin, natural metabolites of human lactoferrin, and functionally equivalent analogues of human apo-lactoferrin;
determining a therapeutically effective amount of the selected substance;
administering the therapeutically effective amount of the selected substance to the patient
whereby the method is used as an alternative to bypass surgery or any therapeutic angiogenesis options.

34. The method as recited in claim 33 whereby the method is used for treating impending stroke, manifested stroke, ischemic heart disease such as angina pectoris or impending or manifested myocardial infarction, and peripheral artery occlusive disease with or without impending gangrene.

35. The method as recited in claim 33 whereby the method is used for treating vascular disease, state of tissue hypoperfusion, or state of depressed VEGF induced angiogenesis associated with peptic ulcer, leg ulcer or local or generalised hair loss.

36. The method as recited in claim 33 wherein said substance is administered orally.

37. The method as recited in claim 33 wherein said substance is administered parenterally.

38. The method as recited in claim 33 wherein said substance is administered locally.

39. The method as recited in claim 33 wherein said substance is administered by inhalation.

40. A method for treatment or prevention of a vascular disease or states of tissue hypoperfusion with hypoxic or ischemic consequences in a patient, the method comprising the following steps:
selecting a peptide from the group consisting of peptides derivable from human lactoferrin;
determining a therapeutically effective amount of the selected substance;
administering the therapeutically effective amount of the selected substance to the patient
whereby the method is used as an alternative to bypass surgery or any therapeutic angiogenesis options.

41. The method as recited in claim 40 wherein the peptide comprises a peptide constituted of all or some of the amino acids 12-40 of human lactoferrin counted from the N-terminal end, or a modified version thereof.

42. The method as recited in claim 40 wherein the peptide comprises a peptide formed of the sequences constituted of amino acids 16-40 and amino acids 18-40 from the N-terminal end of human lactoferrin, or a modified version thereof.

43. The method as recited in claim 40 wherein the peptide comprises a peptide essentially corresponding to residues 18-31 of human lactoferrin wherein C-20 is replaced by A, Q-22 is replaced by K, and N-26 is replaced by D.

44. The method as recited in claim 40 wherein the peptide comprises a peptide formed of the amino acids in positions 12-31, counted from the N-terminal end, in the sequence constituting human lactoferrin, or a modification thereof, or a fragment thereof consisting of at least 7 amino acids.

45. The method as recited in claim 40 wherein the peptide is a peptide consisting of 11-17 amino acids corresponding to the sequences that begin with one of the amino acids in positions 15-21 and end with the amino acid in position 31, counted from the N-terminal end, in the sequence constituting human lactoferrin, or a modification thereof.

46. The method as recited in claim 40 wherein the peptide is a peptide consisting of 12 amino acids based on the sequence consisting of the amino acids in positions 20-31 in human lactoferrin, counted from the N-terminal end.

47. The method as recited in claim 40 whereby the method is used for treating impending stroke, manifested stroke, ischemic heart disease such as angina pectoris or impending or manifested myocardial infarction, and peripheral artery occlusive disease with or without impending gangrene.

48. The method as recited in claim 40 whereby the method is used for treating vascular disease, state of tissue hypoperfusion, or state of depressed VEGF induced angiogenesis associated with peptic ulcer, leg ulcer or local or generalised hair loss.

49. The method as recited in claim 40 wherein said substance is administered orally.

50. The method as recited in claim 40 wherein said substance is administered parenterally.

51. The method as recited in claim 40 wherein said substance is administered locally.

52. The method as recited in claim 40 wherein said substance is administered by inhalation.
